(19) United States
(12) Patent Application Publication
(10) Pub. No.: US 2022/0076801 A1
(43) Pub. Date: Mar. 10, 2022

(54) SYSTEM AND METHOD FOR DETERMINING HUMAN PERFORMANCE

(71) Applicant: UNIVERSITY OF SOUTHERN CALIFORNIA, Los Angeles, CA (US)

(72) Inventors: Peter KUHN, Los Angeles, CA (US); Jorge NIEVA, Los Angeles, CA (US)

(21) Appl. No.: 17/416,382

(22) PCT Filed: Dec. 20, 2019
(86) PCT No.: PCT/US2019/067950

§ 371 Date: Jun. 18, 2021

Related U.S. Application Data
(60) Provisional application No. 62/783,921, filed on Dec. 21, 2018.

(51) Int. Cl. G16H 20/40; A61B 5/11; A61B 5/00; G16H 40/63

(52) U.S. Cl. CPC G16H 20/40; A61B 5/1116; A61B 5/6824; G16H 40/63; A61B 5/4866; A61B 5/4884; A61B 5/1118

(57) ABSTRACT

Human performance is a predictor of survival and response to chemotherapy in patients with cancer. It may be used to initiate new treatment, and monitor patients during ongoing treatment for early signs of deterioration when additional support can still be provided to restore performance and optimize treatment outcomes. This disclosure describes systems and methods for determining whether a cancer patient will need unplanned medical care during cancer therapy (e.g., necessitated by deterioration during cancer therapy). The systems and methods described herein are configured such that the determination is based on an acceleration of patient's center of mass during a prescribed movement, and/or metabolic equivalence determined based on the tracking of a patient's daily activities.

### RELATED PATENT APPLICATION

This patent application is a national phase filing of, and claims the benefit of, International Patent Application No. PCT/US2019/067950, filed on Dec. 20, 2019, entitled “SYSTEM AND METHOD FOR DETERMINING HUMAN PERFORMANCE”, naming Peter Kuhn and Jorge Nieva as inventors, and designated by attorney docket no. 043871-0508992, which claims the benefit of Provisional Patent Application No. 62/783,921 filed on Dec. 21, 2018, entitled “SYSTEM AND METHOD FOR DETERMINING HUMAN PERFORMANCE”, naming Peter Kuhn and Jorge Nieva as inventors, and designated by attorney docket no. 043871-0501304. The entire content of the foregoing patent application is incorporated herein by reference, including all text, tables and drawings.

### FIELD OF THE DISCLOSURE

This disclosure relates to systems and methods for determining human performance. More specifically, this disclosure relates to systems and methods for determining whether a cancer patient will need unplanned medical care during cancer therapy.

### BACKGROUND

Biomechanical characterization of human performance is known. Using biomechanical characterization of human performance to inform decisions about oncological therapy in an effort to reduce or avoid a need for unplanned medical care (e.g., caused by deterioration of a cancer patient) is also known. However, typical biomechanical characterization of human performance for oncological or other reasons often comprises either a qualitative assessment by medical personnel, or an invasive biomechanical characterization test. These require significant experimental setup that includes numerous sensors. In addition, qualitative assessments are difficult to standardize due to their intrinsically subjective nature. Invasive tests provide reliable information but are not feasible for large scale applications.

### SUMMARY

One aspect of the disclosure relates to a system configured to determine whether a cancer patient will need unplanned medical care during cancer therapy. The system comprises one or more sensors, one or more processors, and/or other components. The one or more sensors may be configured to generate output signals conveying spatial position information related to spatial positions of one or more anatomical sites on the cancer patient while the cancer patient performs a prescribed movement. The one or more anatomical sites may comprise an anatomical site that corresponds to a center of mass of the cancer patient, and/or other anatomical sites indicative of mobility of a cancer patient—e.g., a spine base, a knee, a hip, etc.

The one or more processors may be configured to determine one or more kinematic parameters indicative of the movement of the cancer patient during the prescribed movement based on the spatial position information. The one or more kinematic parameters may comprise an acceleration and/or other kinematic parameters of the anatomical site that corresponds to the center of mass of the cancer patient and/or other anatomical sites indicative of mobility. The one or more processors may be configured to determine whether the cancer patient will need unplanned medical care during cancer therapy based on the acceleration and/or other kinematic parameters of the anatomical site that corresponds to the center of mass and/or other anatomical sites indicative of mobility of the cancer patient.

Another aspect of the disclosure relates to a system configured to determine whether a cancer patient will need unplanned medical care during cancer therapy. The system comprises one or more sensors, one or more processors, and/or other components. The one or more sensors may be configured to generate output signals conveying physical activity information related to physical activity performed by the cancer patient. The one or more processors may be configured to determine one or more physical activity parameters indicative of the physical activity of the cancer patient based on the physical activity information. The one or more physical activity parameters may comprise metabolic equivalence (METs). The one or more processors may be configured to determine whether the cancer patient will need unplanned medical care during cancer therapy based on the metabolic equivalence of the cancer patient.

Still another aspect of the disclosure relates to a method for determining whether a cancer patient will need unplanned medical care during cancer therapy with a determination system. The system may comprise one or more sensors, one or more processors, and/or other components. The method comprises generating, with the one or more sensors, output signals conveying spatial position information related to spatial positions of one or more anatomical sites on the cancer patient while the cancer patient performs a prescribed movement. The one or more anatomical sites may comprise an anatomical site that corresponds to a center of mass of the cancer patient and/or other anatomical sites indicative of mobility of the cancer patient. The method may comprise determining, with the one or more processors, one or more kinematic parameters indicative of the movement of the cancer patient during the prescribed movement based on the spatial position information. The one or more kinematic parameters may comprise an acceleration of the anatomical site that corresponds to the center of mass of the cancer patient and/or other kinematic parameters indicative of mobility of the cancer patient. The method may comprise determining, with the one or more processors, whether the cancer patient will need unplanned medical care during cancer therapy based on the acceleration of the anatomical site that corresponds to the center of mass of the cancer patient and/or other kinematic parameters indicative of the mobility of the cancer patient.

Yet another aspect of the disclosure relates to a method for determining whether a cancer patient will need unplanned medical care during cancer therapy with a determination system. The system comprises one or more sensors, one or more processors, and/or other components. The method comprises generating, with the one or more sensors, output signals conveying physical activity information related to physical activity performed by the cancer patient. The method comprises determining, with the one or more processors, one or more physical activity parameters indicative of the physical activity of the cancer patient based on the physical activity information. The one or more physical activity parameters may comprise metabolic equivalence (METs). The method may comprise determining, with the one or more processors, whether the cancer patient will need unplanned medical care during cancer therapy based on the metabolic equivalence of the cancer patient.

It should be noted that, in some embodiments, the patient need not be a cancer patient, and the unplanned medical care may be sought during any future period of time. In some embodiments, the systems and methods described herein may be applied to one or more other cell proliferative disorders, and/or other disorders all together.

These and other objects, features, and characteristics of the system and/or method disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured to determine whether a cancer patient will need unplanned medical care during cancer therapy, in accordance with one or more embodiments.

FIG. 2 illustrates a wire-frame representation of a patient with anatomical sites and corresponding body parts labeled, in accordance with one or more embodiments.

FIG. 3 illustrates a patient performing a prescribed movement associated with a chair to table exam, in accordance with one or more embodiments.

FIG. 4 illustrates a wire frame representation of patient at four different time points during a prescribed movement similar to the prescribed movement shown in FIG. 3, in accordance with one or more embodiments.

FIG. 5 illustrates a time series for the acceleration of the spine base of a cancer patient and a baseline dataset for the same cancer patient, in accordance with one or more embodiments.

FIG. 6 illustrates a method for determining whether a cancer patient will need unplanned medical care during cancer therapy with a determination system, in accordance with one or more embodiments.

### DETAILED DESCRIPTION

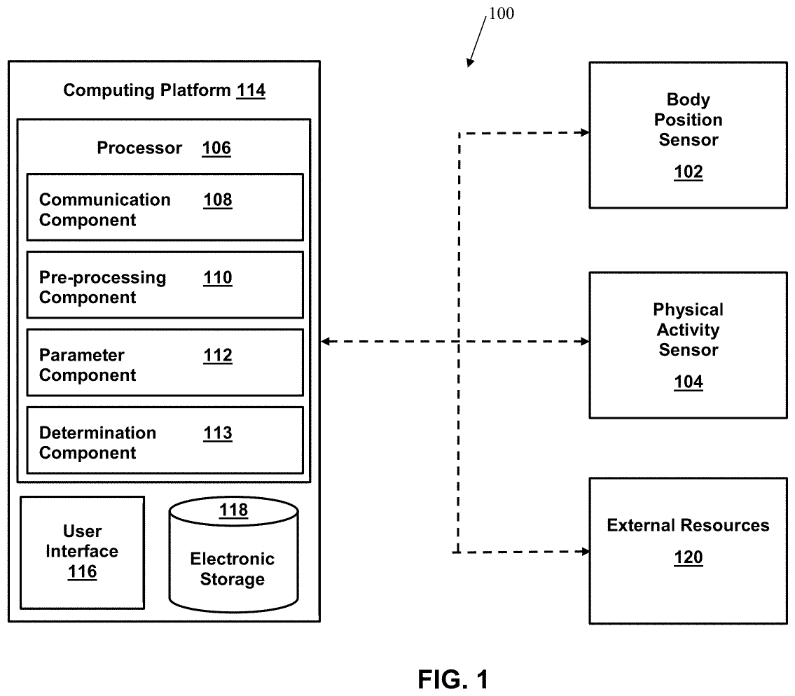

FIG. 1 illustrates a system 100 configured to determine whether a cancer patient will need unplanned medical care during cancer therapy, in accordance with one or more embodiments. Poor patient outcomes, patient satisfaction, quality of life, and economic cost are associated with unplanned medical care for patients actively receiving cancer therapy (e.g., chemotherapy). Predicting a patient's needs during cancer therapy, and providing specific solutions to those needs may improve patient outcomes and the patient's experience during treatment.

Observing the way a patient moves provides a clinician with valuable information about frailty. This is important for patients undergoing difficult treatments such as chemotherapy. A comprehensive geriatric (e.g., frailty) assessment can predict complications and side effects from cancer treatment. However, clinicians' assessments are often qualitative, subjective, and lack agreement among clinicians. Available tools and metrics such as the Eastern Cooperative Oncology Group (ECOG) performance status, body mass index (BMI) measurements, Mini Mental State Exam (MMSE) results, and the Charlson Comorbidity Index (CCI), are often part of a comprehensive geriatric assessment, but few clinicians perform a complete assessment because such assessments are time consuming.

Laboratory based invasive methods have been developed to biomechanically quantify elements of human performance. Many of these methods comprise conducting gait analysis using an accelerometer, a gyroscope, and other types of wearable sensors and motion capture systems to detect and differentiate conditions in patients with osteoarthritis, neuromuscular disorders, and cerebral palsy. However, these methods are associated with high cost, lengthy time required to perform tests, and general difficulty in interpreting results.

Although these tools and metrics are known, and continue to be used because of their practicality, standardization of patient stratification, and speed of assessment; inter- and intra-observer variability, gender discrepancies, sources of subjectivity in physician assigned performance assessments, and a lack of standard conversions between different evaluation scales continue to exist. As such, there is a need for a system and method for more objective classification of a patient's physical function that may be used to guide decisions about oncological therapy in an effort to reduce or avoid a need for unplanned medical care.

Advantageously, system 100 is a non-invasive motion-capture based performance assessment system which can (i) determine kinematic parameters that characterize a cancer patient's biomechanical performance and/or physical activity parameters that characterize a level of physical activity of the cancer patient, and (ii) determine whether a cancer patient will need unplanned medical care during cancer therapy based on the kinematic and/or physical activity parameters. In some embodiments, system 100 comprises one or more of a body position sensor 102; a physical activity sensor 104; computing platform 114 comprising a processor 106, a user interface 116 and electronic storage 118; external resources 120; and/or other components.

Body position sensor 102 may be configured to generate one or more output signals conveying spatial position information and/or other information. The spatial position information and/or other information may be a time series of information that conveys spatial position information about the body and/or body parts of a cancer patient over time. In some embodiments, the spatial position information may comprise visual information representing the body and/or individual body parts of the cancer patient, and/or other information. The visual information representing the cancer patient may include one or more of still images, video images, and/or other information. For example, body position sensor 102 may be configured such that the spatial position information includes body position signals conveying information associated with the position of one or more body parts of the cancer patient relative to each other and/or other reference locations. In some embodiments, the visual information may be and/or include a wire-frame representation of the cancer patient and/or other visual information. According to some embodiments, body position sensor 102 may include an infrared stereoscopic sensor configured to facilitate determination of user body positions, such as for example the KinectlM available from Microsoft™ of Redmond, Wash., and/or other sensors.

Body position sensor 102 may be configured such that the spatial information comprises information associated with one or more body positions and/or other physical characteristics of the cancer patient. The spatial position information in the output signals may be generated responsive to a prescribed movement performed by the cancer patient and/or at other times. A given body position may describe, for example, a spatial position, orientation, posture, and/or other positions of the cancer patient and/or of one or more body parts of the cancer patient. A given physical characteristic may include, for example, a size, a length, a weight, a shape, and/or other characteristics of the cancer patient, and/or of one or more body parts of the cancer patient. The output signals conveying the spatial position information may include measurement information related to the physical size, shape, weight, and/or other physical characteristics of the cancer patient, movement of the body and/or one or more body parts of the cancer patient, and/or other information. The one or more body parts of the cancer patient may include a portion of the first user's body (e.g., one or more of a head, neck, torso, foot, hand, head, arm, leg, and/or other body parts).

The spatial position information may be related to spatial positions of one or more anatomical sites on the cancer patient. The one or more anatomical sites may be and/or correspond to the body parts described above, for example. The one or more anatomical sites may comprise an anatomical site (e.g., a body part) that is indicative of a patient's mobility, corresponds to a center of mass of the cancer patient, and/or include other anatomical sites. In some embodiments, locations that are indicative of a patient's mobility and/or correspond to the center of mass may be a location at a base of a spine of the cancer patient, a location near a hip or hips, a location near a knee, and/or other locations.

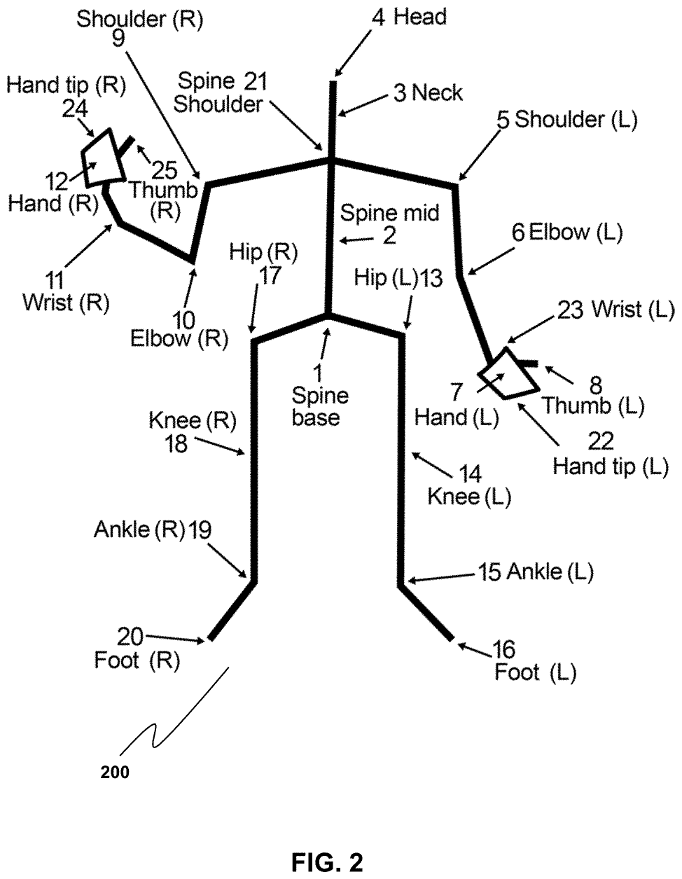

By way of a non-limiting example, FIG. 2 illustrates a wire-frame representation 200 of a patient with anatomical sites 1-20 and corresponding body parts labeled. FIG. 2 illustrates spatial positions of one or more anatomical sites 1-20 on the cancer patient. As described above, the spatial position information in the output signals from body position sensor 102 may comprise visual information representing the body and/or individual body parts of the cancer patient. Wire-frame representation 200 may be and/or be included in such visual information. As shown in FIG. 2, anatomical site 1 corresponds to the base of the patient's spine, anatomical site 2 corresponds to the patient's mid-spine, and so on. Wire frame representation 200 may correspond to a given body position and may describe, for example, a spatial position, orientation, posture, and/or other positions of the cancer patient and/or of one or more body parts of the cancer patient. Wire-frame representation 200 may provide information related to the physical size, shape, weight, and/or other physical characteristics of the cancer patient (e.g., height may represented as a distance from anatomical sites 16 or 20 corresponding to the left or right foot to the anatomical site 4 corresponding to the head), movement of the body and/or one or more body parts of the cancer patient (e.g., movement of anatomical site 1 corresponding to the spine base), relative positions of one or more body parts of the cancer patient, and/or other information. As described above, anatomical site 1, which corresponds to the spine base of the patient, corresponds to a center of mass of the cancer patient. Other anatomical sites indicative of mobility and/or a center of mass of a cancer patient are also contemplated—e.g., a knee, a hip, etc.

The spatial position information (e.g., from body position sensor 102 shown in FIG. 1) may be related to spatial positions of the one or more anatomical sites on the cancer patient while the cancer patient performs the prescribed movement and/or at other times. The prescribed movement may comprise movement associated with a chair to table (CTT) exam, a get up and walk (GUP) exam, and/or other movement, for example.

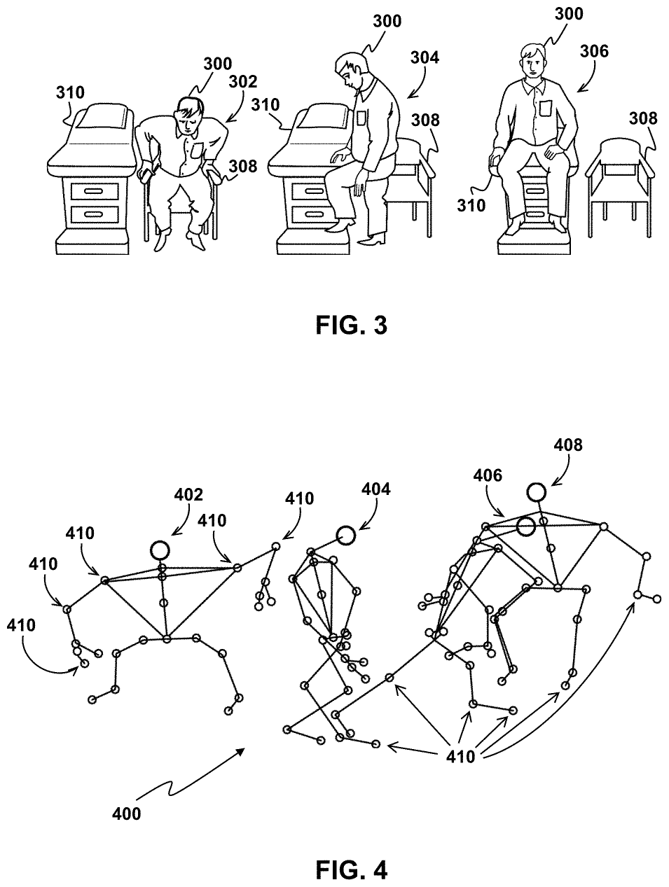

By way of a non-limiting example, FIG. 3 illustrates a patient 300 performing a prescribed movement 302, 304, 306 associated with a chair to table exam. Patient 300 starts in a sitting position in a chair 308 and begins to stand 302. Patient 300 then moves toward, and steps up onto 304 an exam table 310. Patent 300 finishes the prescribed movement by sitting 306 on exam table 310.

FIG. 4 illustrates a wire frame representation 400 of patient (e.g., 300 shown in FIG. 3) at four different time points 402, 404, 406, 408 during a prescribed movement similar to prescribed movement 302, 304, 306 shown in FIG. 3. In FIG. 4, wire frame representation 400 starts in a sitting position (e.g., in a chair that is not shown in FIG. 4) and begins to stand 402, then moves toward 404 and steps up 406 onto an exam table (not shown in FIG. 4), and finishes the prescribed movement by sitting 408 on the exam table. In FIG. 4, wire frame representation 400 is shown moving toward 404 and stepping onto 402 an exam table (not shown in FIG. 4) from the opposite direction shown in FIG. 3. Wire-frame representation 400 illustrates anatomical sites 1-20 illustrated in FIG. 2 as dots 410 at each time point 402, 404, 406, and 408 of the prescribed movement shown in FIG. 4. Wire-frame representation 400 may be and/or be included in the spatial information in the output signals from body position sensor 102 (FIG. 1) described above. Processor 106 (shown in FIG. 1 and described below) may be configured to use wire frame representation 400, for example, and/or other information to determine one or more parameters related to the movement (e.g., a velocity, an acceleration, etc.) of one or more anatomical sites 410. In some embodiments, processor 106 may determine an acceleration of anatomical site 1 (as described herein), which corresponds to the spine base of a cancer patient, and corresponds to a center of mass of the cancer patient. In some embodiments, processor 106 may determine a velocity and/or an acceleration of a knee, a hip, a spine base, and/or other anatomical sites of the cancer patient

Returning to FIG. 1, physical activity sensor 104 may be configured to generate one or more output signals that convey physical activity information and/or other information related to the cancer patient. The physical activity information may be related to physical activity performed by the cancer patient and/or other information. Physical activity performed by the cancer patient may include any movement, motion, and/or other activity performed by the cancer patient. Physical activity may include exercise, normal daily activities, and/or other physical activities. Exercise may include, for example, walking, running, biking, stretching, and/or other exercises. Normal daily activities may include movement through the house, household chores, commuting, working at a computer, shopping, making a meal, and/or other normal daily activities. In some embodiments, physical activity may include maintaining a given posture for a period of time. For example, physical activity may include sitting, standing, lying down, and/or maintaining other postures for a period of time. In some embodiments, physical activity sensor 104 may comprise a wrist worn motion sensor and/or other sensors, for example. In some embodiments, physical activity sensor 104 is and/or includes the Microsoft Band™ available from Microsoft™ of Redmond, Wash., and/or other similar sensors.

In some embodiments, as described above, body position sensor 102 and/or physical activity sensor 104 may be stand-alone devices, separate from one or more other components of system 100, and communicate with one or more other components of system 100 (e.g., computing platform 114) as a peripheral device. In some embodiments, body position sensor 102 and/or physical activity sensor 104 may be integrated with computing platform 114 as a single device (e.g., as a camera that is part of computing platform 114, as an activity tracking sensor built into computing platform 114, etc.). In some embodiments, body position sensor 102, physical activity sensor 104, and/or computing platform 114 may be associated with the cancer patient and/or may be carried by the cancer patient. For example, body position sensor 102 and/or physical activity sensor 104 may be included in a Smartphone associated with the cancer patient. As such, information related to physical activity of the cancer patient may be obtained throughout the day as the cancer patient goes about his daily business and/or participates in specific activities.

Although body position sensor 102 and physical activity sensor 104 are depicted in FIG. 1 as individual elements, this is not intended to be limiting, as other embodiments that include multiple body position sensors 102 and/or physical activity sensors 104 are contemplated and within the scope of the disclosure. For example, in some embodiments, a given computing platform 114 may have one or more integrated body position sensors 102 and/or physical activity sensors 104, and/or be in communication with one or more additional body position sensors 102 and/or physical activity sensors 104 as separate peripheral devices.

Computing platform 114 may include one or more processors 106, a user interface 116, electronic storage 118, and/or other components. Processor 106 may be configured to execute computer program components. The computer program components may be configured to enable an expert or user associated with a given computing platform 114 to interface with system 100 and/or external resources 120, and/or provide other functionality attributed herein to computing platform 114. By way of non-limiting example, computing platform 114 may include one or more of a desktop computer, a laptop computer, a handheld computer, a tablet computing platform, a Smartphone, a gaming console, and/or other computing platforms.

Processor 106 is configured to provide information-processing capabilities in computing platform 114 (and/or system 100 as a whole). As such, processor 106 may comprise one or more of a digital processor, an analog processor, a digital circuit designed to process information, an analog circuit designed to process information, a state machine, and/or other mechanisms for electronically processing information. Although processor 106 is shown in FIG. 1 as a single entity, this is for illustrative purposes only. In some embodiments, processor 106 may comprise a plurality of processing units. These processing units may be physically located within the same device (e.g., computing platform 114), or processor 106 may represent processing functionality of a plurality of devices operating in coordination (e.g., a processor included in computing platform 114, a processor included in body position sensor 102, a processor included in physical activity sensor 104, etc.). In some embodiments, processor 106 may be and/or be included in a computing device such as computing platform 114 (e.g., as described herein). Processor 106 may run one or more electronic applications having graphical user interfaces configured to facilitate user interaction with system 100.

As shown in FIG. 1, processor 106 is configured to execute one or more computer program components. The computer program components may comprise software programs and/or algorithms coded and/or otherwise embedded in processor 106, for example. The computer program components may include one or more of a communication component 108, a pre-processing component 110, a parameter component 112, a determination component 113, and/or other modules. Processor 106 may be configured to execute components 108, 110, 112, and/or 113 by software; hardware; firmware; some combination of software, hardware, and/or firmware; and/or other mechanisms for configuring processing capabilities on processor 106.

It should be appreciated that although components 108, 110, 112, and 113 are illustrated in FIG. 1 as being co-located in processor 106, one or more of the components 108, 110, 112, or 113 may be located remotely from the other components. The description of the functionality provided by the different components 108, 110, 112, and/or 113 described below is for illustrative purposes, and is not intended to be limiting, as any of the components 108, 110, 112, and/or 113 may provide more or less functionality than is described, which is not to imply that other descriptions are limiting. For example, one or more of the components 108, 110, 112, and/or 113 may be eliminated, and some or all of its functionality may be provided by others of the components 108, 110, 112, and/or 113. As another example, processor 106 may include one or more additional components that may perform some or all of the functionality attributed below to one of the components 108, 110, 112, and/or 113.

Communication component 108 may be configured to facilitate bi-directional communication between computing platform 114 and one or more other components of system 100. In some embodiments, the bi-directional communication may facilitate control over one or more of the other components of system 100, facilitate the transfer of information between components of system 100, and/or facilitate other operations. For example, communication component 108 may facilitate control over body position sensor 102 and/or physical activity sensor 104 by a user (e.g., the cancer patient, a doctor, a nurse, a caregiver, etc.). The control may be based on entries and/or selections made by the user via user interface 116, for example, and/or based on other information. As another example, communication component 108 may facilitate uploading and/or downloading data to or from body position sensor 102, physical activity sensor 104, external resources 120, and/or other components of system 10.

Continuing with this example, communication component 108 may be configured to receive the spatial information and/or the physical activity information in the output signals from body position sensor 102 and/or physical activity sensor 104. The output signals may be received directly and/or indirectly from body position sensor 102 and/or physical activity sensor 104. For example, body position sensor 102 may be built into computing platform 114, and the output signals from body position sensor 102 may be transmitted directly to communication component 108. As another example, physical activity sensor 104 may be a separate wrist worn device. The output signals from the wrist worn device may be wirelessly transmitted to communication component 108.

In some embodiments, communication component 108 may be configured to cause display (e.g., on user interface 116) of the spatial information, the physical activity information, a determination, and/or other information. In some embodiments, communication component 108 may be configured to cause display (e.g., on user interface 116) of a graphical control interface to facilitate user control of body position sensor 102, physical activity sensor 104, and/or other components of system 100.

Pre-processing component 110 is configured to pre-process the spatial information, the physical activity information, and/or other information received by communication component 108. In some embodiments, pre-processing comprises filtering, converting, normalizing, adjusting, and/or other pre-processing operations performed on the spatial information, the physical activity information, and/or other information in the output signals from body position sensor 102, physical activity sensor 104, and/or other components of system 100. In some embodiments, pre-processing component 110 may be configured to automatically segment (and/or facilitate manually segmenting) the spatial information to trim irrelevant data at the beginning and end of a prescribed movement while a patient is stationary. Preprocessing component 110 may be configured to pre-process the spatial information to compensate for irregularities in the spatial information caused by the positioning of body position sensor 102 relative to a given cancer patient, features of an environment or location where the prescribed movement occurs, and/or other factors. In some embodiments, pre-processing component 110 may be configured such that pre-processing includes coordinate transformation for three-dimensional data coordinates included in the spatial information. For example, the spatial information received by communication component 108 may be distorted such that a level plane such as a clinic floor appears sloped in the spatial information, for example. In this example, the angle of distortion, 8, may range between about 5 and about 20°. Pre-processing component 110 may be configured to resolve this distortion by performing an automated element rotation about an x-axis of the spatial information. As other examples, in some embodiments, pre-processing may include filters to remove other background humans from the images prior to analysis during the CTT exam; and, for a wrist worn sensor (e.g., as described herein), pre-processing may include adjustments for weight, gender, race, time, diet, and location prior to calculation of metabolic equivalents.

Parameter component 112 may be configured to determine one or more kinematic parameters, physical activity parameters, and/or other parameters. Parameter component 112 may be configured to determine the one or more kinematic and/or physical activity parameters based on the information in the output signals from body position sensor 102 and/or physical activity sensor 104, the pre-processing performed by pre-processing component 110, and/or other information. In some embodiments, the one or more determined kinematic and/or physical activity parameters may be features extracted from the spatial position or physical activity information, and/or other parameters. In some embodiments, the determined kinematic and/or physical activity parameters may comprise less bytes of data than the spatial position information and/or the physical activity information conveyed by the one or more output signals.

In some embodiments, parameter component 112 may be configured to determine one or more kinematic parameters indicative of the movement of the cancer patient during the prescribed movement based on the spatial position information and/or other information. The one or more kinematic parameters may comprise one or more positions of a given anatomical site (e.g., 1-20 shown in FIG. 2) over time, velocities of anatomical sites during the prescribed movement, accelerations (e.g., in any direction) of anatomical sites during the prescribed movement, kinetic energies, potential energies, sagittal angles, and/or other kinematic parameters. For example, parameter component 112 may be configured to determine an acceleration (in any direction) of an anatomical site that corresponds to the center of mass of the cancer patient and/or other parameters. In some embodiments, parameter component 112 may be configured to determine relative accelerations (and/or any other motion related parameter) of one or more anatomical sites. For example, parameter component 112 may be configured to determine a first acceleration of a first anatomical site relative to one or more second accelerations of one or more second anatomical sites. In some embodiments, parameter component 112 may be configured to determine acceleration of an anatomical site relative to a reference site (e.g., an exam table, a patient bed, a computer, and/or other reference sites).

In some embodiments, determining the one or more kinematic parameters indicative of the movement of the cancer patient during the prescribed movement based on the spatial position information comprises determining anatomical site position vectors for the one or more anatomical sites. The anatomical site position vectors may comprise three-dimensional time series generated for given positions of the one or more anatomical sites at time points (e.g., 402, 404, 406, 408 shown in FIG. 4) during the prescribed movement. This may also include determining accelerations for the one or more anatomical sites based on the anatomical site position vectors using a mean-value theorem. For example, parameter component 112 may be configured such that the acceleration of the spine base (e.g., anatomical site 1 shown in FIG. 2 that corresponds to the center of mass of the cancer patient) is determined using the mean-value theorem based on the anatomical site position vectors for the spine base. (Other anatomical sites indicative of mobility and/or a center of mass of a cancer patient are also contemplated—e.g., a knee, a hip, etc.)

By way of a non-limiting example, a position vector

\vec{η}(t)= [symbol] xᵢ(t), yᵢ(t), zᵢ(t)

[symbol]

for an anatomical site i may be used to calculate the anatomical site's velocity magnitude,

vᵢ(t)=∥\vec{η}(t)∥

and acceleration magnitude,

aᵢ(t)=∥\vec{rᵢ}(t)∥

using the mean-value theorem. In the absence of distribution of mass information, specific kinetic energy,

keᵢ(t)=½vᵢ²(t)

and specific potential energy

peᵢ(t)=gΔzᵢ=g(zᵢ(t)−zᵢ(t=1))

quantities may be used to describe the energy signature of each anatomical site. Parameter component 112 may be configured such that the sagittal angle, θₛ(t), is defined as the angle formed between the vector originating at the spine base and pointing in the direction of motion, and the vector connecting the anatomical sites for the spine base (e.g., 1 in FIG. 2) and the neck (e.g., 3 in FIG. 2) at each time point t (e.g., 402, 404, 406, 408 shown in FIG. 4).

In some embodiments, parameter component 112 may be configured to determine one or more physical activity parameters indicative of the physical activity of the cancer patient based on the physical activity information and/or other information. In some embodiments, the one or more physical activity parameters may comprise an amount of time a cancer patient engages in physical activity, a level (e.g., low or high, above or below a predetermined threshold level, etc.) of the physical activity, an amount of energy expended during the physical activity, an amount of calories burned during the physical activity, metabolic equivalence (METs) associated with the physical activity, and/or other parameters. In some embodiments, parameter component 112 may be configured to aggregate (e.g., sum, average, etc.), normalize, and/or perform other operations for the one or more physical activity parameters for a given evaluation period (e.g., per hour, per day, per week, for the time between doctor visits, etc.). In some embodiments, parameter component 112 may be configured to aggregate a given physical activity parameter for the evaluation period only for instances of physical activity that breach a predetermined threshold level during the evaluation period.

For example, in some embodiments, parameter component 112 may be configured to determine total (e.g., a summation of) METs associated with physical activity performed by the cancer patient during the evaluation period. In some embodiments, a total number of METs may be an indication of any and all physical activity by a cancer patient during an evaluation period. METs provide an indication of an amount of energy consumed while sitting at rest relative to an amount of energy consumed while performing a physical activity. In some embodiments, METs may be calculated based on a determination of mechanical work completed. One MET, for example, is equal to 1.1622 watts/kg, where a watt of work is equal to the energy required to move an object at constant velocity of one meter/second against a force of one Newton. Acceleration against force may be determined by integration of a directional force vector from a three-axis accelerometer sensor (e.g., as described herein) and correcting for the weight of the wearer, for example.

In some embodiments, parameter component 112 may be configured such that only METs associated with high levels of physical activity (e.g., physical activity that breaches a predetermined threshold level) may be included in the total. In some embodiments, parameter component 112 may be configured to determine total daily, weekly, or monthly active hours above a threshold of, for example, 1.5 METs (light), 3METs (moderate), or 6 METs (vigorous) physical activity. In some embodiments, parameter component 112 may determine a fraction of daytime hours spent in non-sedentary activity. Total distance travelled and steps taken may be alternative measures of activity, for example.

The physical activity parameters determined by parameter component 112, aggregation operations, threshold levels, and/or other characteristics of parameter component 112 may be determined at manufacture of system 100, determined and/or adjusted by a user via user interface 116, and/or determined in other ways.

Determination component 113 may be configured to determine whether a cancer patient will need unplanned medical care. In some embodiments, the determination of whether the cancer patient will need unplanned medical care during cancer therapy is indicative of a future reaction of the cancer patient to chemotherapy and/or radiation during cancer therapy. In some embodiments, the determining may be based on the acceleration (in any direction) of the anatomical site that corresponds to the center of mass of the cancer patient (e.g., the spine base) and/or other information. In some embodiments, determination component 113 may be configured to determine whether the cancer patient will need unplanned medical care during cancer therapy based on relative accelerations (and/or any other motion parameters) of anatomical sites. For example, determination component 113 may be configured to determine whether the cancer patient will need unplanned medical care based on a comparison of a first acceleration of a first anatomical site to one or more second accelerations of one or more second anatomical sites. In some embodiments, determination component 113 may be configured to determine whether a cancer patient will need unplanned medical care based on acceleration of an anatomical site relative to a reference site (e.g., an exam table, a patient bed, a computer, and/or other reference sites).

In some embodiments, the determining may be based on the metabolic equivalence determined for the cancer patient, and/or other information.

In some embodiments, determining whether the cancer patient will need unplanned medical care during cancer therapy may comprise determining whether the cancer patient will need unplanned medical care during a future period of time that corresponds to one or more cancer therapy treatments received by the cancer patient. In some embodiments, the future period of time is about two months and/or other periods of time. This example is not intended to be limiting.

In some embodiments, determination component 113 may be configured such that determining whether the cancer patient will need unplanned medical care comprises comparing the acceleration of the center of mass of the cancer patient to an acceleration threshold, comparing the METs for the cancer patient to a METs threshold, and/or comparing other parameters to other thresholds, and determining the cancer patient will need unplanned medical care during cancer therapy responsive to a breach of one or more of the thresholds. By way of a non-limiting example, in some embodiments, the spine base acceleration threshold may be about one meter per second squared (1 m/s²), and the METs threshold may be about zero waking hours above 1.5METs (these are merely examples). Determination component 113 may be configured such that if the acceleration of the spine base is in breach of (e.g., below in this example) the spine base acceleration threshold, and/or if the METs are in breach of (e.g., below in this example) the METs threshold, the cancer patient is determined to need unplanned medical care. These examples are not intended to be limiting. The thresholds may be any thresholds on any parameters that are indicative of whether the cancer patient will need unplanned medical care during cancer therapy. In some embodiments, the thresholds may be determined at manufacture of system 100, determined and/or adjusted based on entries and/or selections made by a user via user interface 116, learned by determination component 113 (e.g., as described below), and/or determined in other ways.

In some embodiments, determination component 113 may be configured such that determining whether the cancer patient will need unplanned medical care comprises comparing a spine base acceleration (and/or other parameter) time series (e.g., determined as described above) and/or a physical activity (e.g., as indicated by METs) over time dataset to a corresponding baseline and/or reference dataset. In some embodiments, determination component 113 may be configured to determine a distance between the spine base acceleration time series and/or the physical activity over time dataset and the corresponding baseline and/or reference dataset. For example, the time series for a given feature (e.g., the acceleration of the spine base) may be compared to a baseline and/or reference dataset using Euclidean metric dynamic time warping (DTW), which assigns a distance of zero for completely identical series and larger distances for more dissimilar series.

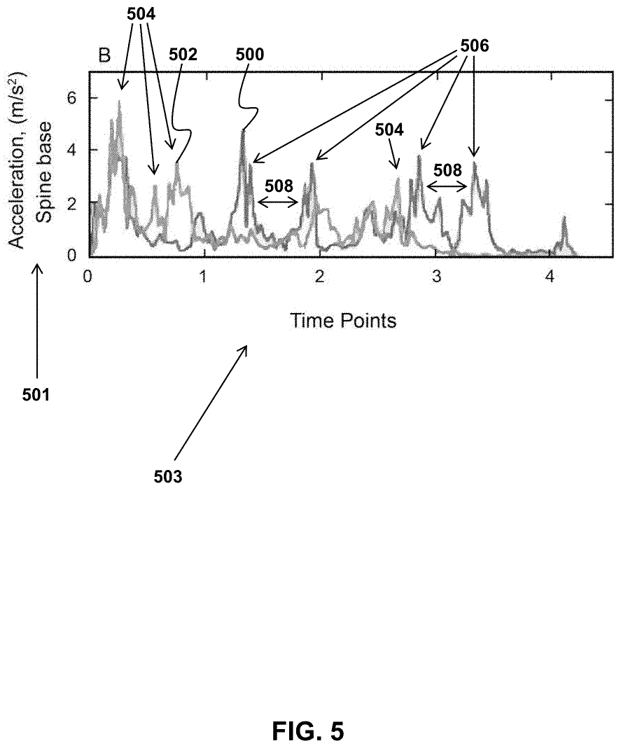

By way of a non-limiting example, FIG. 5 illustrates a time 503 series (e.g., at time points 1, 2, 3, and 4 shown in FIG. 5) 500 for the acceleration 501 of the spine base of a cancer patient and a baseline dataset 502 for the same cancer patient. Determination component 113 may be configured to use DTW to determine a distance between series 500 and 502. Series 500 and series 502 are not the same. They have peaks 504, 506 in different places relative to time points 1-4 and the distances 508 between peaks are not the same, for example. Since series 500 and 502 are not the same, as shown in FIG. 5, DTW would determine a non-zero distance value.

Returning to FIG. 1, determination component 113 may be configured to determine the cancer patient will need unplanned medical care during cancer therapy responsive to a breach of one or more of (DTW) distance thresholds. In some embodiments, the baseline and/or reference datasets, the distance thresholds, and/or other information may be determined at manufacture of system 100, determined and/or adjusted based on entries and/or selections made by a user via user interface 116, learned by determination component 113 (e.g., as described below), and/or determined in other ways.

In some embodiments, determination component 113 is configured to categorize the cancer patient as either likely to likely to need unplanned medical care or unlikely to need unplanned medical care during cancer therapy. In some embodiments, determination component 113 is configured to determine a likelihood (e.g., a numerical value on a continuous scale, a high-medium-low indication, a color representation of the likelihood, etc.) the cancer patient will need unplanned medical care, and categorize the cancer patient into two or more groups based on the likelihood. Determination component 113 may be configured such that the likelihood is inversely correlated to the acceleration of the spine base, the METs, and/or other parameters. For example, higher acceleration of a cancer patient's spine base indicates lower likelihood the cancer patient will need unplanned medical care. Similarly, the higher the number of METs for the cancer patient, the lower the likelihood the cancer patient will need unplanned medical care. In some embodiments, the categorization boundaries, the likelihood determination method, and/or other information may be determined at manufacture of system 100, determined and/or adjusted based on entries and/or selections made by a user via user interface 116, learned by determination component 113 (e.g., as described below), and/or determined in other ways.

In some embodiments, determination component 113 may be configured such that determining whether the cancer patient will need unplanned medical care and/or categorizing the cancer patient as either likely or unlikely to need unplanned medical care may include predicting ECOG scores. In some embodiments, the ECOG scores may be predicted based on the acceleration of the spine base of the cancer patient, the METs associated with the cancer patient, and/or other information, and the determination of whether or not the cancer patient will need unplanned medical care may be based on the ECOG scores.

In some embodiments, determination component 113 may be and/or include a trained prediction model. The trained prediction model may be an empirical model and/or other trained prediction models. The trained prediction model may perform some or all of the operations of determination component 113 described herein. The trained prediction model may predict outputs (e.g., whether or not the cancer patient will need unplanned medical care, ECOG scores, etc.) based on correlations between various inputs (e.g., the spatial information, the physical activity information, etc.).

As an example, the trained prediction model may be a machine learning model. In some embodiments, the machine learning model may be and/or include mathematical equations, algorithms, plots, charts, networks (e.g., neural networks), and/or other tools and machine learning model components. For example, the machine learning model may be and/or include one or more neural networks having an input layer, an output layer, and one or more intermediate or hidden layers. In some embodiments, the one or more neural networks may be and/or include deep neural networks (e.g., neural networks that have one or more intermediate or hidden layers between the input and output layers).

As an example, the one or more neural networks may be based on a large collection of neural units (or artificial neurons). The one or more neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function that combines the values of all its inputs together. In some embodiments, each connection (or the neural unit itself) may have a threshold function such that a signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, the one or more neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for the one or more neural networks may be more free flowing, with connections interacting in a more chaotic and complex fashion. In some embodiments, the intermediate layers of the one or more neural networks include one or more convolutional layers, one or more recurrent layers, and/or other layers.

The machine learning model may be trained (i.e., whose parameters are determined) using a set of training data. The training data may include a set of training samples. The training samples may include spatial information and/or physical activity information, for example, for prior cancer patients, and an indication of whether the prior cancer patients needed unplanned medical care. Each training sample may be a pair comprising an input object (typically a vector, which may be called a feature vector, which may be representative of the spatial and/or physical activity information) and a desired output value (also called the supervisory signal)—for example indicating whether unplanned medical care was needed. A training algorithm analyzes the training data and adjusts the behavior of the machine learning model by adjusting the parameters of the machine learning model based on the training data. For example, given a set of N training samples of the form {(x₁, y₁), (x₂, y₂), . . . , (x_{N}, y_{N})} such that xᵢ is the feature vector of the i-th example and yᵢ is its supervisory signal, a training algorithm seeks a machine learning model g: X→Y, where X is the input space and Y is the output space. A feature vector is an n-dimensional vector of numerical features that represent some object (e.g., the spatial information and/or the physical activity information for a cancer patient as described above). The vector space associated with these vectors is often called the feature space. During training, the machine learning model may learn various parameters such as the spine base acceleration threshold, the METs threshold, the time series distance determination threshold, the categorization boundaries and/or other thresholds as described above. After training, the machine learning model may be used for making predictions using new samples. For example, the trained machine learning model may be configured to predict ECOG scores, whether or not a cancer patient will need unplanned medical care, and/or other information based on corresponding input spatial information and/or physical activity information for the cancer patient.

In some embodiments, determination component 113 may be configured to facilitate adjustment of the cancer therapy and/or other therapies. The adjustment may be based on the determination of whether the patient will need unplanned medical care and/or other information. In some embodiments, facilitating may comprise determining and displaying recommended changes, determining one or more additional parameters from the information in the output signals from the one or more sensors, and/or other operations. For example, based on the determination of whether the patient will need unplanned medical care, in treating a patient with a PD-L1 high expressing lung cancer, an oncologist may choose to treat a patient with a high risk with checkpoint inhibitor therapy alone, rather than a combination of chemotherapy with checkpoint inhibitor therapy. Similarly, a patient with an oral cavity squamous cell carcinoma undergoing combined chemo-radiation may be treated with a lower intensity weekly low-dose cisplatin regimen rather than a higher intensity regimen of high dose cisplatin given at 3 week intervals. Alternatively, physicians may decide to dose reduce chemotherapy to 80% (for example) of the usual standard dose prior to administration of the 1st cycle in anticipation of poor tolerability.

Body position sensor 102, physical activity sensor 104, and processor 106 may be configured to generate, determine, communicate, analyze, present, and/or perform any other operations related to the determinations, the spatial information, the physical activity information and/or any other information in real-time, near real-time, and/or at a later time. For example, the spatial information and/or physical activity information may be stored (e.g., in electronic storage 118) for later analysis (e.g., determination of a prediction). In some embodiments, the stored information may be compared to other previously determined information (e.g., threshold values, etc.), and/or other information.

As shown in FIG. 1, user interface 116 may be configured to provide an interface between computing platform 114 and a user (e.g., a doctor, a nurse, a physical therapy technician, the cancer patient, etc.) through which the user may provide information to and receive information from system 100. This enables data, cues, results, and/or instructions and any other communicable items, collectively referred to as “information,” to be communicated between the user and system 100. Examples of interface devices suitable for inclusion in user interface 116 include a touch screen, a keypad, buttons, switches, a keyboard, knobs, levers, a display, speakers, a microphone, an indicator light, an audible alarm, a printer, and/or other interface devices. In some embodiments, user interface 116 includes a plurality of separate interfaces. In some embodiments, user interface 116 includes at least one interface that is provided integrally with computing platform 114.

It is to be understood that other communication techniques, either hard-wired or wireless, are also contemplated by the present disclosure as user interface 116. For example, the present disclosure contemplates that user interface 116 may be integrated with a removable storage interface provided by computing platform 114. In this example, information may be loaded into computing platform 114 from removable storage (e.g., a smart card, a flash drive, a removable disk) that enables the user to customize the implementation of computing platform 114. Other exemplary input devices and techniques adapted for use with computing platform 114 as user interface 116 include, but are not limited to, an RS-232 port, RF link, an IR link, modem (telephone, cable or other). In short, any technique for communicating information with computing platform 114 and/or system 100 is contemplated by the present disclosure as user interface 116.

Electronic storage 118 may include electronic storage media that electronically stores information. The electronic storage media of electronic storage 118 may include one or both of system storage that is provided integrally (i.e., substantially non-removable) with computing platform 114 and/or removable storage that is removably connectable to computing platform 114 via, for example, a port (e.g., a USB port, a firewire port) or a drive (e.g., a disk drive). Electronic storage 118 may include one or more of optically readable storage media (e.g., optical disks), magnetically readable storage media (e.g., magnetic tape, magnetic hard drive, floppy drive), electrical charge-based storage media (e.g., EEPROM, RAM), solid-state storage media (e.g., flash drive), and/or other electronically readable storage media. Electronic storage 118 may include one or more virtual storage resources (e.g., cloud storage, a virtual private network, and/or other virtual storage resources). Electronic storage 118 may store software algorithms, information determined by processor 106, information received from external resources 120, information entered and/or selected via user interface 116, and/or other information that enables system 100 to function as described herein.

External resources 120 include sources of information such as databases, websites, etc.; external entities participating with system 100 (e.g., systems or networks that store data associated with the cancer patient), one or more servers outside of system 100, a network (e.g., the internet), electronic storage, equipment related to Wi-Fi™ technology, equipment related to Bluetooth® technology, data entry devices, or other resources. In some embodiments, some or all of the functionality attributed herein to external resources 120 may be provided by resources included in system 100. External resources 120 may be configured to communicate with computing platform 114, physical activity sensor 104, body position sensor 102, and/or other components of system 100 via wired and/or wireless connections, via a network (e.g., a local area network and/or the internet), via cellular technology, via Wi-Fi technology, and/or via other resources.

Body position sensor 102, physical activity sensor 104, computing platform 114, and/or external resources 120 may be operatively linked via one or more electronic communication links. For example, such electronic communication links may be established, at least in part, via wires, via local network using Wi-Fi, Bluetooth, and/or other technologies, via a network such as the Internet and/or a cellular network, and/or via other networks. It will be appreciated that this is not intended to be limiting, and that the scope of this disclosure includes embodiments in which body position sensor 102, physical activity sensor 104, computing platform 114, and/or external resources 120 may be operatively linked via some other communication media, or with linkages not shown in FIG. 1. In some embodiments, as described above, computing platform 114, body position sensor 102, physical activity sensor 104, and/or other devices may be integrated as a singular device.

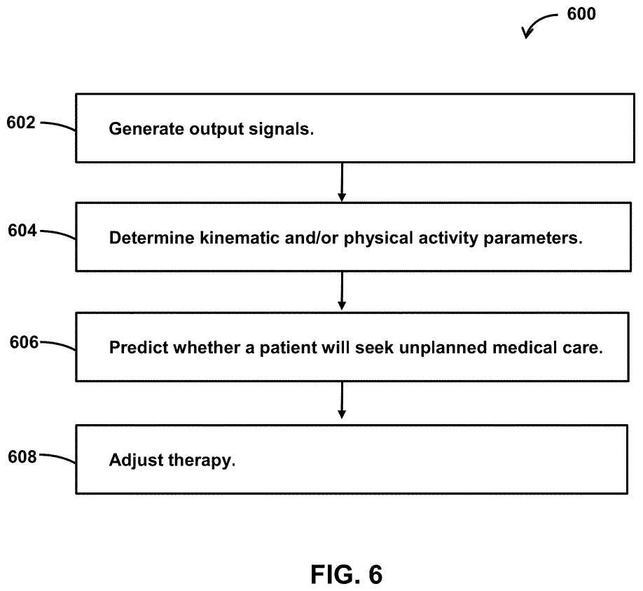

FIG. 6 illustrates a method 600 for determining whether a cancer patient will need unplanned medical care during cancer therapy with a determination system, in accordance with one or more embodiments. Unplanned medical care may comprise medical care unrelated to the cancer therapy, unscheduled medical care, non-routine medical care, emergency medical care, and/or other unplanned medical care. The system comprises one or more sensors, one or more processors, and/or other components. The operations of method 600 presented below are intended to be illustrative. In some embodiments, method 600 may be accomplished with one or more additional operations not described, and/or without one or more of the operations discussed. Additionally, the order in which the operations of method 600 are illustrated in FIG. 6 and described below is not intended to be limiting.

In some embodiments, method 600 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 600 in response to instructions stored electronically on an electronic storage medium. The one or more processing devices may include one or more devices configured through hardware, firmware, and/or software to be specifically designed for execution of one or more of the operations of method 600.

At an operation 602, output signals may be generated. In some embodiments, the output signals may convey spatial position information related to spatial positions of one or more anatomical sites on the cancer patient while the cancer patient performs a prescribed movement. The spatial position information may comprise visual information representing the body of the cancer patient and/or other information. The one or more anatomical sites may comprise an anatomical site that corresponds to a center of mass of the cancer patient. In some embodiments, the one or more anatomical sites may comprise anatomical sites indicative of mobility and/or the center of mass of a cancer patient, and/or other anatomical sites. In some embodiments, a location that corresponds to the center of mass and/or that is indicative of mobility may be a location at a base of a spine of the cancer patient, a location at or near the hips of a cancer patient, locations and/or near the knees of a cancer patient, and/or other locations. The prescribed movement may comprise movement associated with a chair to table (CTT) exam and/or other movement, for example.

In some embodiments, the output signals may convey physical activity information related to physical activity performed by the cancer patient. In these embodiments, the one or more sensors may comprise a wrist worn motion sensor and/or other sensors, for example. In some embodiments, operation 602 may be performed by one or more sensors similar to or the same as body position sensor 102 and/or physical activity sensor 104 (shown in FIG. 1, and described herein).

At an operation 604, kinematic and/or physical activity parameters may be determined. In some embodiments, the one or more determined kinematic and/or physical activity parameters may be features extracted from the spatial position or physical activity information, and/or other parameters. In some embodiments, the determined kinematic and/or physical activity parameters may comprise less bytes of data than the spatial position information and/or the physical activity information conveyed by the one or more output signals. In some embodiments, operation 604 may include determining one or more kinematic parameters indicative of the movement of the cancer patient during the prescribed movement based on the spatial position information and/or other information. The one or more kinematic parameters may comprise velocities, accelerations, and/or other kinematic parameters. For example, the one or more kinematic parameters may comprise an acceleration of an anatomical site that corresponds to the center of mass of the cancer patient, a velocity and/or acceleration of an anatomical site indicative of mobility of the cancer patient, and/or other parameters. In some embodiments, determining the one or more kinematic parameters indicative of the movement of the cancer patient during the prescribed movement based on the spatial position information comprises determining anatomical site position vectors for the one or more anatomical sites. The anatomical site position vectors may comprise three-dimensional time series generated for given positions of the one or more anatomical sites at given time points during the prescribed movement. This may also include determining accelerations for the one or more anatomical sites based on the anatomical site position vectors using a mean-value theorem. The acceleration of an anatomical site that corresponds to the center of mass (for example) of the cancer patient may be determined using the mean-value theorem based on anatomical site position vectors for the anatomical site that corresponds to the center of mass of the cancer patient, for example.

In some embodiments, operation 604 may include determining one or more physical activity parameters indicative of the physical activity of the cancer patient based on the physical activity information and/or other information. In these embodiments, the one or more physical activity parameters may comprise metabolic equivalence (METs) and/or other parameters. In some embodiments, operation 604 may be performed by one or more processors configured to execute a computer program component similar to or the same as parameter component 112 (shown in FIG. 1, and described herein).

Operation 606 may include determining whether a patient will need unplanned medical care. In some embodiments, the determining may be based on an acceleration of an anatomical site that corresponds to the center of mass of the cancer patient, velocities and/or accelerations of anatomical sites indicative of mobility, and/or other information. In some embodiments, the determining may be based on the metabolic equivalence determined for the cancer patient, and/or other information.

In some embodiments, the determination of whether the cancer patient will need unplanned medical care during cancer therapy is indicative of a future reaction of the cancer patient to chemotherapy and/or radiation during cancer therapy. In some embodiments, determining whether the cancer patient will need unplanned medical care during cancer therapy comprises determining whether the cancer patient will need unplanned medical care during a future period of time that corresponds to one or more cancer therapy treatments received by the cancer patient. In some embodiments, the future period of time is about two months and/or other periods of time. In some embodiments, operation 606 comprises categorizing the cancer patient as either likely to likely to need unplanned medical care or unlikely to need unplanned medical care during cancer therapy. In some embodiments, operation 606 comprises determining a likelihood the cancer patient will need unplanned medical care, and categorizing the cancer patient into two or more groups based on the likelihood. In some embodiments, operation 606 may be performed by one or more processors configured to execute a computer program component similar to or the same as determination component 113 (shown in FIG. 1, and described herein).

At an operation 608, therapy may be adjusted. The adjusted therapy may be the cancer therapy and/or other therapies. The adjusting may be based on the determination of whether the patient will need unplanned medical care and/or other information. In some embodiments, adjusting may include facilitating adjustment of the cancer therapy based on the determination of whether the cancer patient will need unplanned medical care during cancer therapy. In some embodiments, facilitating may comprise determining and displaying recommended changes, determining one or more additional parameters from the information in the output signals from the one or more sensors, and/or other operations. In some embodiments, operation 608 may be performed by one or more processors configured to execute a computer program component similar to or the same as determination component 113 (shown in FIG. 1 and described herein).

Although the present technology has been described in detail for the purpose of illustration based on what is currently considered to be the most practical and preferred embodiments, it is to be understood that such detail is solely for that purpose and that the technology is not limited to the disclosed embodiments, but, on the contrary, is intended to cover modifications and equivalent arrangements that are within the spirit and scope of the appended claims. For example, it is to be understood that the present technology contemplates that, to the extent possible, one or more features of any embodiment can be combined with one or more features of any other embodiment.

What is claimed is:

1. A system configured to determine whether a cancer patient will need unplanned medical care during cancer therapy, the system comprising:
one or more sensors configured to generate output signals conveying spatial position information related to spatial positions of one or more anatomical sites on the cancer patient while the cancer patient performs a prescribed movement, the one or more anatomical sites comprising an anatomical site that corresponds to a center of mass of the cancer patient; and
one or more processors configured by machine readable instructions to:
determine one or more kinematic parameters indicative of the movement of the cancer patient during the prescribed movement based on the spatial position information, the one or more kinematic parameters comprising an acceleration of the anatomical site that corresponds to the center of mass of the cancer patient; and
determine whether the cancer patient will need unplanned medical care during cancer therapy based on the acceleration of the anatomical site that corresponds to the center of mass of the cancer patient.

2. The system of claim 1, wherein the one or more sensors are configured such that the location that corresponds to the center of mass is a location at a base of a spine of the cancer patient.

3. The system of claim 1, wherein the one or more processors are configured such that determining the one or more kinematic parameters indicative of the movement of the cancer patient during the prescribed movement based on the spatial position information comprises:
determining anatomical site position vectors for the one or more anatomical sites, the anatomical site position vectors comprising three-dimensional time series generated for given positions of the one or more anatomical sites at given time points during the prescribed movement; and
determining accelerations for the one or more anatomical sites based on the anatomical site position vectors using a mean-value theorem, such that the acceleration of the anatomical site that corresponds to the center of mass of the cancer patient is determined using the mean-value theorem based on anatomical site position vectors for the anatomical site that corresponds to the center of mass of the cancer patient.

4. The system of clam 1, wherein the prescribed movement comprises movement associated with a chair to table (CTT) exam.

5. The system of claim 1, wherein unplanned medical care comprises one or more of medical care unrelated to the cancer therapy, unscheduled medical care, non-routine medical care, or emergency medical care.

6. The system of claim 1, wherein the one or more processors are further configured to facilitate adjustment of the cancer therapy based on the determination of whether the cancer patient will need unplanned medical care during cancer therapy.

7. The system of claim 1, wherein the one or more processors are configured such that the determination of whether the cancer patient will need unplanned medical care during cancer therapy is indicative of a future reaction of the cancer patient to chemotherapy and/or radiation during cancer therapy.

8. The system of claim 1, wherein the one or more processors are configured such that determining whether the cancer patient will need unplanned medical care during cancer therapy comprises determining whether the cancer patient will need unplanned medical care during a future period of time that corresponds to one or more cancer therapy treatments received by the cancer patient.

9. The system of claim 8, wherein the one or more processors are configured such that the future period of time is about two months.

10. The system of claim 1, wherein the one or more processors are further configured to categorize the cancer patient as either likely to likely to need unplanned medical care or unlikely to need unplanned medical care during cancer therapy, wherein the categorization comprises predicting Eastern Cooperative Oncology Group (ECOG) scores.

11. The system of claim 1, wherein the one or more processors are configured such that determining whether the cancer patient will need unplanned medical care during cancer therapy based on the acceleration of the anatomical site that corresponds to the center of mass of the cancer patient comprises determining a likelihood the cancer patient will need unplanned medical care, and categorizing the cancer patient into two or more groups based on the likelihood, the likelihood comprising a numerical value on a continuous scale, the likelihood being inversely correlated to acceleration of the anatomical site that corresponds to the center of mass of the cancer patient.

12. The system of claim 1, wherein the one or more sensors are configured such that the spatial position information comprises visual information representing the body of the cancer patient.

13. The system of claim 1, wherein the one or more processors are configured such that the one or more determined kinematic parameters comprise less bytes of data than the spatial position information conveyed by the one or more output signals.

14. The system of claim 1, wherein the one or more processors are configured such that determining whether the cancer patient will need unplanned medical care during cancer therapy based on the acceleration of the anatomical site that corresponds to the center of mass of the cancer patient comprises comparing the acceleration of the anatomical side that corresponds to the center of mass of the cancer patient to a corresponding acceleration threshold, and determining the cancer patient will need unplanned medical care during cancer therapy responsive to a breach.

15. The system of claim 1, wherein the one or more processors are configured such that determining whether the cancer patient will need unplanned medical care comprises comparing a spine base acceleration time series to a corresponding baseline, determining a distance between the spine base acceleration time series and the corresponding baseline using Euclidean metric dynamic time warping (DTW), which assigns a distance of zero for completely identical series and larger distances for more dissimilar series, and determining the cancer patient will need unplanned medical care during cancer therapy responsive to a breach of one or more DTW distance thresholds.

16. A system configured to determine whether a patient will need unplanned medical care during a future period of time, the system comprising:
one or more sensors configured to generate output signals conveying spatial position information related to spatial positions of one or more anatomical sites on the patient while the patient performs a prescribed movement, the one or more anatomical sites comprising an anatomical site that corresponds to a center of mass of the patient; and
one or more processors configured by machine readable instructions to:
determine one or more kinematic parameters indicative of the movement of the patient during the prescribed movement based on the spatial position information, the one or more kinematic parameters comprising an acceleration of the anatomical site that corresponds to the center of mass of the patient; and
determine whether the patient will need unplanned medical care during the future period of time based on the acceleration of the anatomical site that corresponds to the center of mass of the patient.

17. A system configured to determine whether a cancer patient will need unplanned medical care during cancer therapy, the system comprising one or more processors configured by machine readable instructions to:
receive output signals from one or more sensors conveying spatial position information related to spatial positions of one or more anatomical sites on the cancer patient while the cancer patient performs a prescribed movement, the one or more anatomical sites comprising an anatomical site that corresponds to a center of mass of the cancer patient;
determine one or more kinematic parameters indicative of the movement of the cancer patient during the prescribed movement based on the spatial position information, the one or more kinematic parameters comprising an acceleration of the anatomical site that corresponds to the center of mass of the cancer patient; and
determine whether the cancer patient will need unplanned medical care during cancer therapy based on the acceleration of the anatomical site that corresponds to the center of mass of the cancer patient.

18. A system configured to determine whether a cancer patient will need unplanned medical care during cancer therapy, the system comprising:
one or more sensors configured to generate output signals conveying physical activity information related to physical activity performed by the cancer patient; and
one or more processors configured by machine readable instructions to:
determine one or more physical activity parameters indicative of the physical activity of the cancer patient based on the physical activity information, the one or more physical activity parameters comprising metabolic equivalence (METs); and
determine whether the cancer patient will need unplanned medical care during cancer therapy based on the metabolic equivalence of the cancer patient.

19. The system of claim 18, wherein the one or more sensors comprise a wrist worn motion sensor.

20. The system of claim 18, wherein unplanned medical care comprises one or more of medical care unrelated to the cancer therapy, unscheduled medical care, non-routine medical care, or emergency medical care.

21. The system of claim 18, wherein the one or more processors are further configured to facilitate adjustment of the cancer therapy based on the determination of whether the cancer patient will need unplanned medical care during cancer therapy.

22. The system of claim 18, wherein the one or more processors are configured such that the determination of whether the cancer patient will need unplanned medical care curing cancer therapy is indicative of a future reaction of the cancer patient to chemotherapy and/or radiation during cancer therapy.

23. The system of claim 18, wherein the one or more processors are configured such that determining whether the cancer patient will need unplanned medical care during cancer therapy comprises determining whether the cancer patient will need unplanned medical care during a future period of time that corresponds to one or more cancer therapy treatments received by the cancer patient.

24. The system of claim 18, wherein the one or more processors are further configured to categorize the cancer patient as either likely to likely to need unplanned medical care or unlikely to need unplanned medical care during cancer therapy, wherein the categorization comprises predicting Eastern Cooperative Oncology Group (ECOG) scores.

25. The system of claim 18, wherein the one or more processors are configured such that determining whether the cancer patient will need unplanned medical care during cancer therapy comprises determining a likelihood the cancer patient will need unplanned medical care, and categorizing the cancer patient into two or more groups based on the likelihood, the likelihood comprising a numerical value on a continuous scale, the likelihood being inversely correlated to the metabolic equivalence of the cancer patient.

26. The system of claim 18, wherein the one or more processors are configured such that determining whether the cancer patient will need unplanned medical care during cancer therapy based on the metabolic equivalence of the patient comprises comparing the metabolic equivalence of the patient to a corresponding metabolic equivalence threshold, and determining the cancer patient will need unplanned medical care during cancer therapy responsive to a breach.

27. The system of claim 18, wherein the one or more processors are configured such that determining whether the cancer patient will need unplanned medical care comprises comparing a metabolic equivalence over time dataset to a corresponding reference dataset, determining a distance between the metabolic equivalence over time dataset and the corresponding reference dataset using Euclidean metric dynamic time warping (DTW), which assigns a distance of zero for completely identical series and larger distances for more dissimilar series, and determining the cancer patient will need unplanned medical care during cancer therapy responsive to a breach of one or more DTW distance thresholds.

28. A system configured to determine whether a patient will need unplanned medical care during a future period of time, the system comprising:
one or more sensors configured to generate output signals conveying physical activity information related to physical activity performed by the patient; and
one or more processors configured by machine readable instructions to:
determine one or more physical activity parameters indicative of the physical activity of the patient based on the physical activity information, the one or more physical activity parameters comprising metabolic equivalence (METs); and
determine whether the patient will need unplanned medical care during the future period of time based on the metabolic equivalence of the patient.

29. A system configured to determine whether a cancer patient will need unplanned medical care during cancer therapy, the system comprising one or more processors configured by machine readable instructions to:
receive output signals from one or more sensors conveying physical activity information related to physical activity performed by the cancer patient;
determine one or more physical activity parameters indicative of the physical activity of the cancer patient based on the physical activity information, the one or more physical activity parameters comprising metabolic equivalence (METs); and
determine whether the cancer patient will need unplanned medical care during cancer therapy based on the metabolic equivalence of the cancer patient.

30. A method for determining whether a cancer patient will need unplanned medical care during cancer therapy with a determination system, the system comprising one or more sensors and one or more processors, the method comprising:
generating, with the one or more sensors, output signals conveying spatial position information related to spatial positions of one or more anatomical sites on the cancer patient while the cancer patient performs a prescribed movement, the one or more anatomical sites comprising an anatomical site that corresponds to a center of mass of the cancer patient;
determining, with the one or more processors, one or more kinematic parameters indicative of the movement of the cancer patient during the prescribed movement based on the spatial position information, the one or more kinematic parameters comprising an acceleration of the anatomical site that corresponds to the center of mass of the cancer patient; and
determining, with the one or more processors, whether the cancer patient will need unplanned medical care during cancer therapy based on the acceleration of the anatomical site that corresponds to the center of mass of the cancer patient.

31. The method of claim 30, wherein the location that corresponds to the center of mass is a location at a base of a spine of the cancer patient.

32. The method of claim 30, wherein determining the one or more kinematic parameters indicative of the movement of the cancer patient during the prescribed movement based on the spatial position information comprises:
determining anatomical site position vectors for the one or more anatomical sites, the anatomical site position vectors comprising three-dimensional time series generated for given positions of the one or more anatomical sites at given time points during the prescribed movement; and
determining accelerations for the one or more anatomical sites based on the anatomical site position vectors using a mean-value theorem, such that the acceleration of the anatomical site that corresponds to the center of mass of the cancer patient is determined using the mean-value theorem based on anatomical site position vectors for the anatomical site that corresponds to the center of mass of the cancer patient.

33. The method of clam 30, wherein the prescribed movement comprises movement associated with a chair to table (CTT) exam.

34. The method of claim 30, wherein unplanned medical care comprises one or more of medical care unrelated to the cancer therapy, unscheduled medical care, non-routine medical care, or emergency medical care.

35. The method of claim 30, further comprising facilitating, with the one or more processors, adjustment of the cancer therapy based on the determination of whether the cancer patient will need unplanned medical care during cancer therapy.

36. The method of claim 30, wherein the determination of whether the cancer patient will need unplanned medical care curing cancer therapy is indicative of a future reaction of the cancer patient to chemotherapy and/or radiation during cancer therapy.

37. The method of claim 30, wherein determining whether the cancer patient will need unplanned medical care during cancer therapy comprises determining whether the cancer patient will need unplanned medical care during a future period of time that corresponds to one or more cancer therapy treatments received by the cancer patient.

38. The method of claim 37, wherein the future period of time is about two months.

39. The method of claim 30, further comprising categorizing, with the one or more processors, the cancer patient as either likely to likely to need unplanned medical care or unlikely to need unplanned medical care during cancer therapy, wherein the categorization comprises predicting Eastern Cooperative Oncology Group (ECOG) scores.

40. The method of claim 30, wherein determining whether the cancer patient will need unplanned medical care during cancer therapy based on the acceleration of the anatomical site that corresponds to the center of mass of the cancer patient comprises determining a likelihood the cancer patient will need unplanned medical care, and categorizing the cancer patient into two or more groups based on the likelihood, the likelihood comprising a numerical value on a continuous scale, the likelihood being inversely correlated to acceleration of the anatomical site that corresponds to the center of mass of the cancer patient.

41. The method of claim 30, wherein the spatial position information comprises visual information representing the body of the cancer patient.

42. The method of claim 30, wherein the one or more determined kinematic parameters comprise less bytes of data than the spatial position information conveyed by the one or more output signals.

43. The method of claim 30, wherein determining whether the cancer patient will need unplanned medical care during cancer therapy based on the acceleration of the anatomical site that corresponds to the center of mass of the cancer patient comprises comparing the acceleration of the anatomical side that corresponds to the center of mass of the cancer patient to a corresponding acceleration threshold, and determining the cancer patient will need unplanned medical care during cancer therapy responsive to a breach.

44. The method of claim 30, wherein determining whether the cancer patient will need unplanned medical care comprises comparing a spine base acceleration time series to a corresponding baseline, determining a distance between the spine base acceleration time series and the corresponding baseline using Euclidean metric dynamic time warping (DTW), which assigns a distance of zero for completely identical series and larger distances for more dissimilar series, and determining the cancer patient will need unplanned medical care during cancer therapy responsive to a breach of one or more DTW distance thresholds.

45. A method for determining whether a patient will need unplanned medical care during a future period of time with a determination system, the system comprising one or more sensors and one or more processors, the method comprising:
generating, with the one or more sensors, output signals conveying spatial position information related to spatial positions of one or more anatomical sites on the patient while the patient performs a prescribed movement, the one or more anatomical sites comprising an anatomical site that corresponds to a center of mass of the patient;
determining, with the one or more processors, one or more kinematic parameters indicative of the movement of the patient during the prescribed movement based on the spatial position information, the one or more kinematic parameters comprising an acceleration of the anatomical site that corresponds to the center of mass of the patient; and
determining, with the one or more processors, whether the patient will need unplanned medical care during the future period of time based on the acceleration of the anatomical site that corresponds to the center of mass of the patient.

46. A method for determining whether a cancer patient will need unplanned medical care during cancer therapy with a determination system, the system comprising one or more processors, the method comprising:
receiving, with the one or more processors, output signals from one or more sensors conveying spatial position information related to spatial positions of one or more anatomical sites on the cancer patient while the cancer patient performs a prescribed movement, the one or more anatomical sites comprising an anatomical site that corresponds to a center of mass of the cancer patient;
determining, with the one or more processors, one or more kinematic parameters indicative of the movement of the cancer patient during the prescribed movement based on the spatial position information, the one or more kinematic parameters comprising an acceleration of the anatomical site that corresponds to the center of mass of the cancer patient; and
determining, with the one or more processors, whether the cancer patient will need unplanned medical care during cancer therapy based on the acceleration of the anatomical site that corresponds to the center of mass of the cancer patient.

47. A method for determining whether a cancer patient will need unplanned medical care during cancer therapy with a determination system, the system comprising one or more sensors and one or more processors, the method comprising:
generating, with the one or more sensors, output signals conveying physical activity information related to physical activity performed by the cancer patient;
determining, with the one or more processors, one or more physical activity parameters indicative of the physical activity of the cancer patient based on the physical activity information, the one or more physical activity parameters comprising metabolic equivalence (METs); and
determining, with the one or more processors, whether the cancer patient will need unplanned medical care during cancer therapy based on the metabolic equivalence of the cancer patient.

48. The method of claim 47, wherein the one or more sensors comprise a wrist worn motion sensor.

49. The method of claim 47, wherein unplanned medical care comprises one or more of medical care unrelated to the cancer therapy, unscheduled medical care, non-routine medical care, or emergency medical care.

50. The method of claim 47, further comprising facilitating, with the one or more processors, adjustment of the cancer therapy based on the determination of whether the cancer patient will need unplanned medical care during cancer therapy.

51. The method of claim 47, wherein the determination of whether the cancer patient will need unplanned medical care curing cancer therapy is indicative of a future reaction of the cancer patient to chemotherapy and/or radiation during cancer therapy.

52. The method of claim 47, wherein determining whether the cancer patient will need unplanned medical care during cancer therapy comprises determining whether the cancer patient will need unplanned medical care during a future period of time that corresponds to one or more cancer therapy treatments received by the cancer patient.

53. The method of claim 47, further comprising categorizing, with the one or more processors, the cancer patient as either likely to likely to need unplanned medical care or unlikely to need unplanned medical care during cancer therapy, wherein the categorization comprises predicting Eastern Cooperative Oncology Group (ECOG) scores.

54. The method of claim 47, wherein determining whether the cancer patient will need unplanned medical care during cancer therapy comprises determining a likelihood the cancer patient will need unplanned medical care, and categorizing the cancer patient into two or more groups based on the likelihood, the likelihood comprising a numerical value on a continuous scale, the likelihood being inversely correlated to the metabolic equivalence of the cancer patient.

55. The method of claim 47, wherein determining whether the cancer patient will need unplanned medical care during cancer therapy based on the metabolic equivalence of the patient comprises comparing the metabolic equivalence of the patient to a corresponding metabolic equivalence threshold, and determining the cancer patient will need unplanned medical care during cancer therapy responsive to a breach.

56. The method of claim 47, wherein determining whether the cancer patient will need unplanned medical care comprises comparing a metabolic equivalence over time dataset to a corresponding reference dataset, determining a distance between the metabolic equivalence over time dataset and the corresponding reference dataset using Euclidean metric dynamic time warping (DTW), which assigns a distance of zero for completely identical series and larger distances for more dissimilar series, and determining the cancer patient will need unplanned medical care during cancer therapy responsive to a breach of one or more DTW distance thresholds.

57. A method for determining whether a patient will need unplanned medical care during a future period of time with a determination system, the system comprising one or more sensors and one or more processors, the method comprising:
generating, with the one or more sensors, output signals conveying physical activity information related to physical activity performed by the patient;
determining, with the one or more processors, one or more physical activity parameters indicative of the physical activity of the patient based on the physical activity information, the one or more physical activity parameters comprising metabolic equivalence (METs); and
determining, with the one or more processors, whether the patient will need unplanned medical care during the future period of time based on the metabolic equivalence of the patient.

58. A method for determining whether a cancer patient will need unplanned medical care during cancer therapy with a determination system, the system comprising one or more processors, the method comprising:
receiving, with the one or more processors, output signals from one or more sensors conveying physical activity information related to physical activity performed by the cancer patient;
determining, with the one or more processors, one or more physical activity parameters indicative of the physical activity of the cancer patient based on the physical activity information, the one or more physical activity parameters comprising metabolic equivalence (METs); and
determining, with the one or more processors, whether the cancer patient will need unplanned medical care during cancer therapy based on the metabolic equivalence of the cancer patient.

59. A system configured to determine whether a cancer patient will need unplanned medical care during cancer therapy, the system comprising:
one or more sensors configured to generate output signals conveying spatial position information related to spatial positions of one or more anatomical sites on the cancer patient while the cancer patient performs a prescribed movement; and
one or more processors configured by machine readable instructions to:
determine one or more kinematic parameters indicative of the movement of the cancer patient during the prescribed movement based on the spatial position information, the one or more kinematic parameters comprising a velocity and/or an acceleration of a knee, a hip, and/or a spine base of the cancer patient; and
determine whether the cancer patient will need unplanned medical care during cancer therapy based on the velocity and/or the acceleration of the knee, the hip, and/or the spine base of the cancer patient.

60. A method for determining whether a cancer patient will need unplanned medical care during cancer therapy with a determination system, the system comprising one or more sensors and one or more processors, the method comprising:
generating, with the one or more sensors, output signals conveying spatial position information related to spatial positions of one or more anatomical sites on the cancer patient while the cancer patient performs a prescribed movement;
determining, with the one or more processors, one or more kinematic parameters indicative of the movement of the cancer patient during the prescribed movement based on the spatial position information, the one or more kinematic parameters comprising a velocity and/or an acceleration of a knee, a hip, and/or a spine base of the cancer patient; and
determining, with the one or more processors, whether the cancer patient will need unplanned medical care during cancer therapy based on the velocity and/or the acceleration of the knee, the hip, and/or the spine base of the cancer patient.

61. A system configured to determine whether a cancer patient will need unplanned medical care during cancer therapy, the system comprising:
one or more sensors configured to generate output signals conveying spatial position information related to spatial positions of one or more anatomical sites on the cancer patient while the cancer patient performs a prescribed movement; and
one or more processors configured by machine readable instructions to:
determine one or more kinematic parameters indicative of the movement of the cancer patient during the prescribed movement based on the spatial position information, the one or more kinematic parameters comprising an acceleration of an anatomical site; and
determine whether the cancer patient will need unplanned medical care during cancer therapy based on the acceleration of the anatomical site.

62. A method for determining whether a cancer patient will need unplanned medical care during cancer therapy with a determination system, the system comprising one or more sensors and one or more processors, the method comprising:
generating, with the one or more sensors, output signals conveying spatial position information related to spatial positions of one or more anatomical sites on the cancer patient while the cancer patient performs a prescribed movement;
determining, with the one or more processors, one or more kinematic parameters indicative of the movement of the cancer patient during the prescribed movement based on the spatial position information, the one or more kinematic parameters comprising an acceleration of an anatomical site; and
determining, with the one or more processors, whether the cancer patient will need unplanned medical care during cancer therapy based on the acceleration of the anatomical site.

63. A system configured to determine whether a cancer patient will need unplanned medical care during cancer therapy, the system comprising:
one or more sensors configured to generate output signals conveying spatial position information related to spatial positions of one or more anatomical sites on the cancer patient while the cancer patient performs a prescribed movement; and
one or more processors configured by machine readable instructions to:
determine one or more kinematic parameters indicative of the movement of the cancer patient during the prescribed movement based on the spatial position information, the one or more kinematic parameters comprising accelerations of the one or more anatomical sites; and
determine whether the cancer patient will need unplanned medical care during cancer therapy based on a comparison of a first acceleration of a first anatomical site to one or more second accelerations of one or more second anatomical sites.

64. A method for determining whether a cancer patient will need unplanned medical care during cancer therapy with a determination system, the system comprising one or more sensors and one or more processors, the method comprising:
generating, with the one or more sensors, output signals conveying spatial position information related to spatial positions of one or more anatomical sites on the cancer patient while the cancer patient performs a prescribed movement;
determining, with the one or more processors, one or more kinematic parameters indicative of the movement of the cancer patient during the prescribed movement based on the spatial position information, the one or more kinematic parameters comprising accelerations of the one or more anatomical sites; and
determining, with the one or more processors, whether the cancer patient will need unplanned medical care during cancer therapy based on a comparison of a first acceleration of a first anatomical site to one or more second accelerations of one or more second anatomical sites.

65. A system configured to determine whether a cancer patient will need unplanned medical care during cancer therapy, the system comprising:
one or more sensors configured to generate output signals conveying spatial position information related to spatial positions of one or more anatomical sites on the cancer patient while the cancer patient performs a prescribed movement and a reference site unrelated to the one or more anatomical sites on the cancer patient; and
one or more processors configured by machine readable instructions to:
determine one or more kinematic parameters indicative of the movement of the cancer patient relative to the reference site during the prescribed movement based on the spatial position information, the one or more kinematic parameters comprising accelerations of the one or more anatomical sites; and
determine whether the cancer patient will need unplanned medical care during cancer therapy based on an acceleration of an anatomical site relative to the reference site.

66. The system of claim 62, wherein the reference site comprises one or more of an exam table, a patient bed, or a computer.

67. A method for determining whether a cancer patient will need unplanned medical care during cancer therapy with a determination system, the system comprising one or more sensors and one or more processors, the method comprising:
generating, with the one or more sensors, output signals conveying spatial position information related to spatial positions of one or more anatomical sites on the cancer patient while the cancer patient performs a prescribed movement and a reference site unrelated to the one or more anatomical sites on the cancer patient;
determining, with the one or more processors, one or more kinematic parameters indicative of the movement of the cancer patient relative to the reference site during the prescribed movement based on the spatial position information, the one or more kinematic parameters comprising accelerations of the one or more anatomical sites; and
determining, with the one or more processors, whether the cancer patient will need unplanned medical care during cancer therapy based on an acceleration of an anatomical site relative to the reference site.

68. The method of claim 67, wherein the reference site comprises one or more of an exam table, a patient bed, or a computer.
